(19) United States
(12) Patent Application Publication
(10) Pub. No.: US 2008/0107748 A1
(43) Pub. Date: May 8, 2008

(54) Microcapsules for removing and absorbing a chemical from a mixture

(76) Inventor: Krishan Kumar Singal, Millersville, MD (US)

Correspondence Address: Nathaniel Gordon-Clark, 1025 North Calvert Street, Baltimore, MD, 21202, US

(21) Appl. No.: 11/982,917

(22) Filed: Nov. 5, 2007

Related U.S. Application Data
(60) Provisional application No. 60/857,102, filed on Nov. 6, 2006.

(51) Int. Cl. A61K 9/50

(52) U.S. Cl. 424/491; 424/490; 424/493

(57) ABSTRACT

A microcapsule comprising a chemical skeleton with at least one ligand attached to the chemical skeleton. The chemical skeleton is placed within a capsule. The capsule can then separate a substance from a mixture. The capsule is placed within the mixture and the substance is allowed to enter the capsule. The substance binds to the ligand and cannot escape the capsule. The capsule is then removed from the mixture, thus removing the substance. The preferred embodiment envisions the use of the capsule to remove substances from animal digestive systems.

### CROSS-REFERENCE TO RELATED APPLICATIONS

This application claims the benefit of Provisional Patent Application Ser. No. 60/857102, filed Nov. 6, 2006 by the present inventor.

### FEDERALLY SPONSORED RESEARCH

None

### SEQUENCE LISTING OR PROGRAM

None

### FIELD OF INVENTION

This invention relates to microcapsules that can remove a specific substance from a mixture. More specifically it relates to microcapsules that can remove a specific substance from an animals body. Even more specifically it relates to microcapsules that can remove a specific substance from a human body.

### BACKGROUND OF THE INVENTION

The pharmaceutical Xenical prevents the body from absorbing fat. Unfortunately, the fat molecules remain in the chyme in the intestinal lumen and are digested by native bacteria. These bacteria digest the unabsorbed fat into gases. These gases produce odorous flatulence. At the same time, the undigested fats result in incontinence. Clearly, these are unpleasant side effects of Xenical.

The solution is to remove the fat molecules from the chyme so that the bacteria do not digest the fat molecules. Thus a substance needs to be put into the chyme that competitively absorbs the fat from the chyme. Such a substance could actually replace Xenical, because absorbing the fat would not only prevent the native bacteria from digesting the fat, but would also prevent the body from absorbing the fat. Thus such a substance would both cure the side effects of Xenical and be a diet aid in of itself.

### SUMMARY OF INVENTION

The invention is a method and a product for absorbing material from a liquid or semi-liquid mixture. The envisioned use of the invention is to remove material from the intestinal lumen of an animal. The preferred embodiment envisions the use of the invention to remove nutrients such as carbohydrates and fats from the intestine of a person. The invention could be used on any animal. The invention could be used in any fluid medium and any organ. The invention could be used to absorb any material. The invention could be used with any mixture.

The invention is a microcapsule that contains a chemical skeleton inside the microcapsule. Both the microcapsule and the skeleton are insoluble in the mixture that the invention will be used in. Both the microcapsule and the skeleton will not dissolve, react or corrode in the mixture that the invention is used in. The skeleton has one or more types of ligands attached to it. The number of ligands is one or more for each type of ligand. The ligand is selected for the ability to bond with the material in the mixture that the invention is intended to absorb from the mixture.

The microcapsule can be of any material. The microcapsule can allow materials from the mixture to diffuse through the microcapsule. The microcapsule can have pores. The pores can be of any size. The pores can allow material from the mixture to enter the microcapsule. The pores should be large enough to allow the material to diffuse into the microcapsule but small enough to prevent the ligand from diffusing out of the microcapsule and to prevent enzymes from diffusing into the microcapsule.

The preferred embodiment of the invention envisions a plurality of microcapsules each with a plurality of pores. The preferred embodiment envisions microcapsules that are larger than a red blood cell so that the microcapsules cannot enter the bloodstream through a capillary. The skeleton within the microcapsule has a plurality of ligands for lipids and components of lipid molecules. Components of lipid molecules include glycerides and fatty acids. In the intestine of the user, the invention will absorb lipids and the components of lipid molecules by binding the ligand to the lipids and components of lipid molecules. Thus, the invention will trap the lipids and components of lipid molecules within the microcapsule. The microcapsule will then pass through the digestive system of the user, removing the lipids and components of lipid molecules from the user's body. The goal of the invention in the preferred embodiment is to absorb lipids and the components of lipids before the user's body can absorb these molecules and then remove the molecules form the user's body through the normal excretion function of the digestive system.

The invention can be delivered in to a body by any means effective. The invention is intended to be swallowed by a user. The invention then travels to the intestine of the user. Possible means of the delivery include pills, capsules, powders, pastes, solutions, suspensions and chewable food items. The preferred embodiment of the invention envisions a pill made of a plurality of microcapsules. A pill used to deliver the microcapsules of the invention may be bound together by inactive binding material. Alternatively, a pill used to deliver the microcapsules may have a digestible shell. The user swallows the pill. The microcapsules disperse before the microcapsules enter the intestinal lumen of the user, or disperse once the microcapsules arrive in the intestinal lumen of the user.

In the preferred embodiment, the invention relies on diffusion to move the material from the mixture into the microcapsules. The preferred embodiment, envisions the creation of a diffusion gradient of that material. Thus the material is encouraged to diffuse into the microcapsules. The invention maintains this diffusion gradient by having the material bind to the ligand. This prevents the inside of the microcapsule from developing an increased concentration of the material, because the material inside the microcapsule is bound to the ligand. Thus even as material diffuses into the microcapsule, the diffusion gradient is maintained and the material continues to diffuse into the microcapsule.

The material that the invention could absorb could be any material. The preferred embodiment envisions the absorption of lipids and the components of lipid molecules. The invention could also be used to absorb other nutrients, for example, and not meant as a limitation in any way, carbohydrates, amino acids and Sodium etc. The invention could also be used to absorb other chemicals produced by the body, for example, and not meant as a limitation in any way, enzymes, urea or insulin. The invention could also be used to absorb toxins and other harmful agents. The invention could also be used to absorb pharmaceutical compounds. The invention could also be used to absorb the byproducts of a pharmaceutical. Thus the invention could both be used as a diet aid and as a poison and drug overdose cure.

### BRIEF DESCRIPTION OF THE DRAWINGS

The accompanying drawings, which are incorporated in and form part of the specification, illustrate the embodiments of the present invention and, together with the description, serve to explain the principles of the invention.

FIG. 1 is a picture of a cross section of a microcapsule.

FIG. 2 is a picture of a microcapsule in the intestinal lumen with molecules of a material.

### DETAILED DESCRIPTION OF THE DRAWINGS

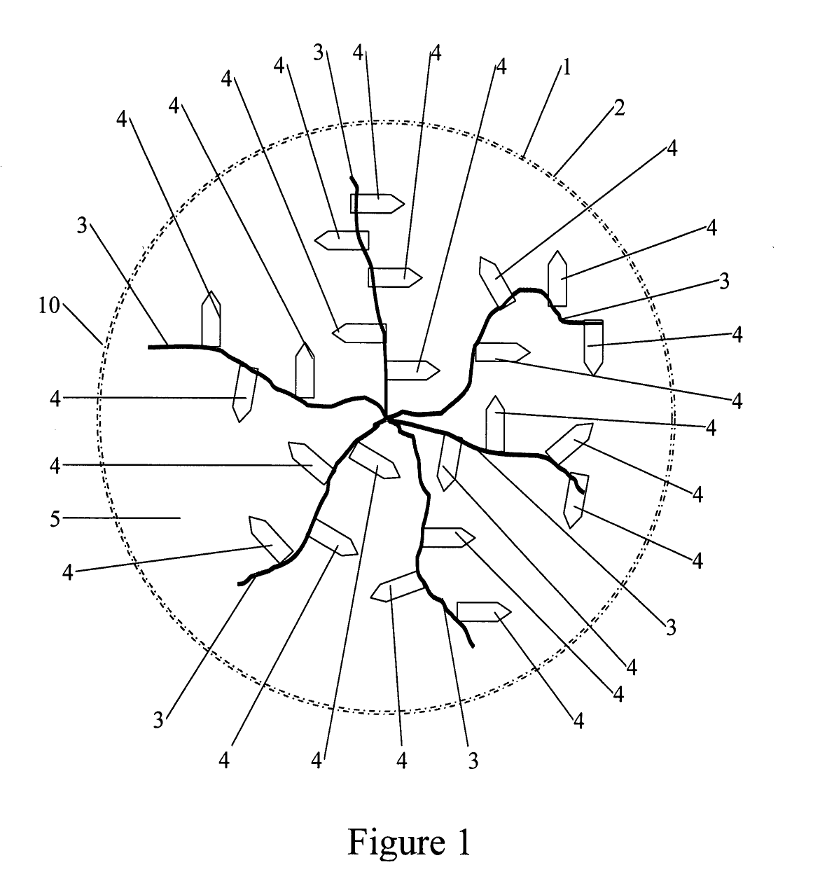

FIG. 1 illustrates a cross section of a microcapsule. The microcapsule 1 has a membrane 10 with pores 2. Inside the microcapsule 1 is the interior 5 and the skeleton 3. Attached to the skeleton 3 is a plurality of ligands 4.

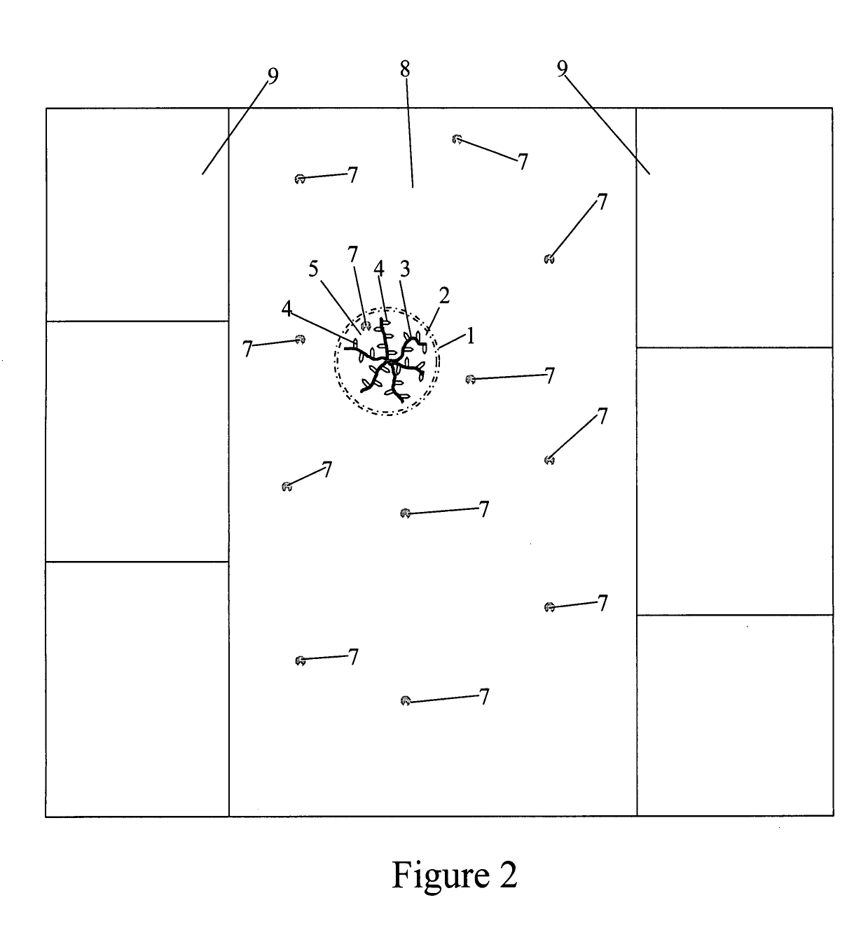

FIG. 2 illustrates a microcapsule in the intestinal lumen with molecules of a material. The microcapsule 1 has a membrane 10 with pores 2. Inside the microcapsule 1 is the interior 5 and the skeleton 3. Attached to the skeleton 3 is a plurality of ligands 4. In this figure, the majority of the ligands 4 are not labeled. The microcapsule 1 is inside the intestinal lumen 8. The intestinal walls 9 define the intestinal lumen 8. Inside the intestinal lumen 8 are molecules 7 of a material. The figure also shows a captured molecule 6 that has diffused into the interior 5 of the microcapsule 1 and bound to a ligand 4.

The microcapsule 1 can be of any size and any shape. In the preferred embodiment, the microcapsule 1 should be larger than a red blood cell so that the microcapsule can not enter the capillaries. In the preferred embodiment, the microcapsule 1 should be around 10 micrometers. The microcapsule 1 must not be digestible by the solution that microcapsule 1 is used in. In the preferred embodiment, the microcapsule 1 must not be digestible by humans. Also, in the preferred embodiment, the microcapsule 1 must not be harmful to humans. In embodiments of the microcapsule 1 where the microcapsule 1 is used in a water based solution, the microcapsule must be water insoluble. In the preferred embodiment, the microcapsule 1 is bio-degradable.

The pore 2 can be of any size. The pore 2 should be of a size that the desired molecule 7 of material can diffuse freely into the microcapsule 1. The pore 2 should be of a size that does not allow the ligand 4 to diffuse out of the microcapsule 1. The pore 2 should be of a size that does not allow enzymes to diffuse into the microcapsule 1.

The ligand 4 can be one type of ligand 4 or more than one type of ligand 4. The number of ligands 4 can be one or more than one. The ligand 4 is selected for its ability to bond to the desired molecule 7 of the material. The ligand 4 must not be able to diffuse out of the microcapsule 1. The ligand 4 can be attached to skeleton 3 so that ligand 4 will not dissolve or diffuse out. Alternatively, ligand 4 can be too large to fit through pore 2. In the preferred embodiment, the ligand 4 should bind with lipids. In another preferred embodiment, the ligand 4 should bind with fatty acids. In another preferred embodiment, the ligand 4 should bind with glycerides. In another preferred embodiment, the ligand 4 should bind with fat. In another preferred embodiment, the ligand could be an ion exchange resin or other type of exchange chemical.

Although this invention has been illustrated by reference to specific embodiments, it will be apparent to those skilled in the art that various changes and modification may be made which clearly fall within the scope of the invention. The invention is intended to be protected broadly within the spirit and scope of the appended claims.

What is claimed is:

1. A microcapsule comprising:
a chemical skeleton;
a capsule with an inside portion;
a ligand;
a substance that will bind to the ligand;
where the chemical skeleton is within the inside portion of the capsule and the ligand is bound to the skeleton;
so that when the microcapsule is exposed to the substance, the substance enters the capsule and binds to the ligand.

2. The microcapsule of claim 1 where the substance is a lipid.

3. The microcapsule of claim 1 where the substance is a carbohydrate.

4. The microcapsule of claim 1 where the substance is a protein.

5. The microcapsule of claim 1 where the substance is insulin.

6. The microcapsule of claim 1 where the substance is a toxin.

7. A microcapsule created by a method comprising:
preparing a chemical skeleton;
preparing a capsule;
attaching at least one ligand to the chemical skeleton;
placing the chemical skeleton within the capsule;
so that when the microcapsule is exposed to a substance that will bind to the ligand, the substance binds to the ligand and becomes trapped within the capsule.

8. The microcapsule of claim 7 where the substance is a lipid.

9. The microcapsule of claim 7 where the substance is a carbohydrate.

10. The microcapsule of claim 7 where the substance is a protein.

11. The microcapsule of claim 7 where the substance is insulin.

12. The microcapsule of claim 7 where the substance is a toxin.

13. A method of using a microcapsule to remove a substance from the digestive system of an animal comprising:
preparing a chemical skeleton;
preparing a capsule;
attaching at least one ligand to the chemical skeleton;
placing the chemical skeleton within the capsule;
so that when the microcapsule is ingested by an animal, the microcapsule enters the digestive system of the animal that contains a substance that will bind to the ligand, and the microcapsule is exposed to a substance that will bind to the ligand, then the substance binds to the ligand and becomes trapped within the capsule and the animal's body then excretes the microcapsule and removes the microcapsule and the substance trapped in the microcapsule.

14. The microcapsule of claim 13 where the substance is a toxin.

15. The microcapsule of claim 13 where the substance is a lipid.

16. The microcapsule of claim 13 where the substance is insulin.

17. The microcapsule of claim 1 where the capsule includes pores in the capsule to allow a substance to diffuse into and out off the capsule.

18. The microcapsule of claim 7 where the capsule includes pores in the capsule to allow a substance to diffuse into and out off the capsule.

19. The microcapsule of claim 13 where the capsule includes pores in the capsule to allow a substance to diffuse into and out off the capsule.

20. A microcapsule comprising a microcapsule for absorbing/binding and then removing a chemical from a solution.

21. A microcapsule according to claim 20 that will not get digested/disintegrated in human intestinal tract.

22. A microcapsule according to claim 20 wherein said microcapsule has pores that allow free diffusion of the molecules to be removed from the solution.

23. A microcapsule according to claim 20 wherein said microcapsule has pores that do not allow enzymes to enter the microcapsule.

24. A microcapsule according to claim 20 wherein said microcapsule can absorb/bind nutrients in digestive tract.

25. A microcapsule according to claim 20 where the core ligand has specific property of binding specific chemicals, ions, molecules or part thereof.

26. A microcapsule according to claim 20 wherein the core ligand is attached to a skeleton or otherwise processed so it cannot leach out of the microcapsule.
